(19) United States
(12) Patent Application Publication
(10) Pub. No.: US 2024/0058092 A1
(43) Pub. Date: Feb. 22, 2024

(54) BIOPSY SITE MARKER HAVING EXPANDABLE PORTION

(71) Applicant: Devicor Medical Products, Inc., Cincinnati, OH (US)

(72) Inventors: Garrett A. HOUSEHOLDER, Cincinnati, OH (US); Jack A. RANDALL, Cincinnati, OH (US); Xavier ROBIN, Karlsruhe (DE); Steffen MUELLER, Gernsbach (DE); Bernd VOGEL, Karlsruhe (DE)

(21) Appl. No.: 18/231,921

(22) Filed: Aug. 9, 2023

Related U.S. Application Data
(60) Provisional application No. 63/398,333, filed on Aug. 16, 2022.

(51) Int. Cl. A61B 90/00

(52) U.S. Cl. CPC A61B 90/39; A61B 2090/3987; A61B 2090/3991; A61B 2090/3908

(57) ABSTRACT

A biopsy site marker includes a marker element. The marker element includes a base portion and an anchor portion. The anchor portion extends distally from the base portion. The anchor portion includes a plurality of arms. Each arm of the plurality of arms is configured to be responsive to heat to transition from a pre-deployment configuration to a post-deployment configuration. Each arm of the plurality of arms extends outwardly from a longitudinal axis defined by the marker element when the plurality of arms are in the post-deployment configuration.

### PRIORITY

This application claims priority to U.S. Provisional Application Ser. No. 63/398,333, entitled “Biopsy Site Marker Having Expandable Portion,” filed on Aug. 16, 2022, the disclosure of which is incorporated by reference herein.

### BACKGROUND

Breast biopsies may be performed because of irregular mammograms and palpable abnormalities. Biopsies can include surgical excisional biopsies and stereotactic and ultrasound guided needle breast biopsies. In the case of image directed biopsy, the radiologist or other physician may take a small sample of the irregular tissue for laboratory analysis. If the biopsy proves to be malignant, additional surgery (e.g., a lumpectomy or a mastectomy) may be required. In the case of needle biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers used after breast biopsies to mark the location where the biopsied tissue was removed is described in the following US patents: U.S. Pat. No. 6,083,524, “Polymerizable biodegradable polymers including carbonate or dioxanone linkages,” issued Jul. 4, 2000; U.S. Pat. No. 6,162,241, “Hemostatic tissue sealants,” issued Dec. 4, 2000; U.S. Pat. No. 6,270,464, “Biopsy localization method and device,” issued Aug. 7, 2001; U.S. Pat. No. 6,356,782, “Subcutaneous cavity marking device and method,” issued Mar. 12, 2002; U.S. Pat. No. 6,605,294, “Methods of using in situ hydration of hydrogel articles for sealing or augmentation of tissue or vessels,” issued Aug. 12, 2003; U.S. Pat. No. 8,600,481, “Subcutaneous cavity marking device,” issued Dec. 3, 2013 and U.S. Pat. No. 8,939,910, “Method for enhancing ultrasound visibility of hyperechoic materials”, issued Jan. 27, 2015. All of these US patents are incorporated by reference in their entirety.

Once a marker is placed at a biopsy site, the marker can later be relocated to identify the biopsy site in subsequent follow-up procedures. In some contexts, a placed marker may not completely correspond to the biopsy site when the marker is relocated. For instance, the marker may migrate to another nearby location during the intervening time between the biopsy procedure and subsequent follow up procedures. Migration of the biopsy site marker may cause difficulties when identifying the biopsy site during subsequent follow-up procedures. Accordingly, it may be desirable to incorporate features into a marker to maintain the marker in a fixed position over time.

While several systems and methods have been made and used for marking a biopsy site, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view an exemplary biopsy site marker;

FIG. 2 depicts a detailed side elevational view of the marker of FIG. 1;

FIG. 3A depicts a side elevational view of the marker of FIG. 1, the marker being in a pre-deployment configuration;

FIG. 3B depicts another side elevational view of the marker of FIG. 1, the marker being in a post-deployment configuration;

FIG. 4 depicts a front elevational view of the marker of FIG. 1, the marker being in the post-deployment configuration of FIG. 3B; and

FIGS. 5A, 5B, and 5C show exemplary aspects of placement of another biopsy site marker, in accordance with aspects of the present disclosure;

FIG. 6 depicts a perspective view of an exemplary marker delivery device;

FIG. 7 depicts a side cross-sectional view of the marker delivery device of FIG. 6; and

FIG. 8 depicts a cross-sectional view of a marker being deployed from the distal portion of the marker delivery device of FIG. 6 and through a lateral aperture in a biopsy needle to mark a biopsy site.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification.

Once a marker is positioned at a biopsy site, it may be desirable for the marker to remain visible under ultrasound. It may also be desirable to make the marker readily identifiable relative to other structural features of a patient. For instance, it may be desirable for the marker to be distinguishable under ultrasound visualization from microcalcifications to avoid inadvertently characterizing the marker as a microcalcification during subsequent ultrasonic examinations. Generally, microcalcifications are used in the field to identify suspicious lesions or masses. Thus, it is generally desirable for the ultrasound view to be distinguishable as a marker and not inadvertently identified as a new mass.

### I. EXEMPLARY BIOPSY SITE MARKER FOR LIMITED MIGRATION

In some versions of a biopsy site marker it may be desirable to include certain features within the marker to reduce the propensity of the marker to migrate when placed within tissue. For instance, some markers may be prone to migration after placement at a biopsy site due to movement of tissue in the intervening time between marker placement and subsequent follow-up procedures. As a result, such markers may introduce challenges with accurately identifying the biopsy site during subsequent follow-up procedures. Accordingly, it may be desirable to incorporate features into a marker to assist in maintaining the marker in a fixed position within tissue over time. Although several versions are described herein that incorporate the features outlined below, it should be understood that various alternative combinations can be used without departing from the basic principles described herein.

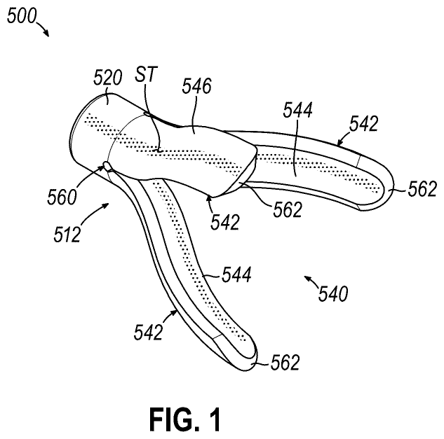

FIG. 1 shows an exemplary marker (500) that is generally configured to expand from a pre-deployment configuration to a post-deployment configuration to thereby anchor marker (500) within tissue. In some versions, marker (500) is similar to marker (100) described in greater detail below. For instance, like with marker (100), marker (500) of the present version includes a marker element (512). In some versions, marker element (512) can be substantially similar to marker element (12) described below. Thus, marker element (512) can be generally configured as non-bioabsorbable and radiopaque and/or echogenic to enhance visualization over time. Additionally, although not shown, it should be understood that in some versions, marker (500) may include structures similar to carrier (120) described in greater detail below. In versions including structures similar to carrier (120), such structures may be shaped as described below or alternatively configured as a coating. In other versions, marker (500) may only include marker element (512), omitting structures similar to carrier (120) described below. In other words, in some versions, marker (500) may be configured as a “bare” marker.

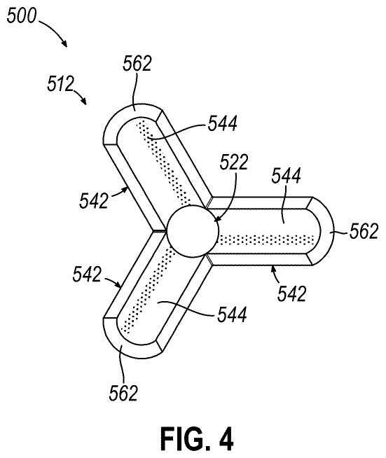

As noted above, marker (500) of the present version is configured to transition from a pre-deployment configuration to a post-deployment configuration after being deployed at a biopsy site. Marker (500) includes a base portion (520) and an anchor portion (540), with both base portion (520) and anchor portion (540) being defined by marker element (512). Base portion (520) defines a generally cylindrical shape. In the present version, base portion (520) is hollow and defines an open proximal end (522) (see FIG. 4). In other versions, base portion (520) may be partially or substantially solid.

All edges of base portion (520) may be atraumatic in structure. In other words, base portion (520) generally includes no sharp edges or corners. Thus, at the intersection of any given surface of base portion (e.g., distal end and the cylindrical exterior) may be rounded, chamfered, eased, beveled or otherwise softened to smooth the profile of such intersections.

Anchor portion (540) extends distally from base portion (520) with the hollow interior of base portion (520) being open to the interior of anchor portion (540). As will be described in greater detail below, anchor portion (540) is generally configured to transition from a pre-deployment configuration to a post-deployment configuration in response to one or more environmental stimuli.

Although reference to “anchor” in anchor portion (540) herein may suggest that only anchor portion (540) may serve to anchor marker (500) within tissue, it should be understood that other features of marker (500) may also be used to facilitate anchoring in tissue. Such anchoring functionality may be present even if a feature is not referred to herein directly using the term “anchor” or similar terms. For instance, under some circumstances, the open proximal end (522) of base portion (520) may also have an anchoring function by facilitating the growth of tissue into marker (500). In addition, or in the alternative, some versions of marker (500) may include structures similar to carrier (120) (described below), which may expand within tissue and serve to further facilitate anchoring of marker (500) within tissue.

Anchor portion (540) of the present version includes a plurality of arms (542) oriented in a circular pattern. Although the present version includes three arms (542), it should be understood that in other versions other suitable numbers of arms (542) may be used such as two, four, five, etc. Each arm (542) is defined by a plurality of slots extending from the distal end of marker element (512) to base portion. As will be described in greater detail below, arms (542) are generally configured to move from a pre-deployment position to a post-deployment position. For reference, the post-deployment position is shown in FIG. 1.

Each arm (542) defines a generally semi-cylindrical cross-sectional shape such that each arm (542) may be abutted with each adjacent arm (542) to form a cylindrical shape corresponding to the shape of base portion (520). In particular, each arm (542) includes a curved inner surface (544) and a curved outer surface (546). Thus, when each arm (542) is positioned to abut each adjacent arm (542), curved surfaces (544, 546) together form a cylindrical inner and outer surface.

The proximal end of each arm (542) is secured to the distal end of base portion (520) such that each arm (542) extends distally from the distal end of base portion (520). In the present version, each arm (542) is integral with base portion (520), although in other versions, each arm (542) may be fixedly secured to base portion (520).

Base portion (520) and/or a portion of each arm (542) defines a plurality of relief openings (560). Each relief opening (560) defines a generally tear-dropped or round-shaped configuration. Each relief opening (560) further extends entirely through the surface of base portion (520) and/or a portion of each arm (542) into a hollow interior of marker element (512). As will be described in greater detail below, each relief opening (560) is configured to promote flexion of each arm (542) relative to base portion (520) and each adjacent arm (542).

The distal end of each arm (542) includes a distal tip (562) having a blunt configuration. In particular, all edges of each arm (542) intersecting at distal tip (562) are generally rounded or otherwise blended together such that distal tip (562) of each arm (542) is generally atraumatic in configuration. Such a configuration may be desirable in some versions to promote movement of arms (542) through tissue either during deployment or after deployment. For instance, during deployment, the blunt configuration of each distal tip (562) may be desirable to promote ease of movement through various instruments, devices, and/or tissue. Similarly, after deployment, the blunt configuration of each distal tip (562) may promote movement arms (542) through tissue so that movement is not impeded and surrounding tissue is not damaged unnecessarily.

In the present version, each arm (542) is substantially identical to all other arms (542) aside from the position of each arm (542) relative to base portion (520). It should be understood that in other versions, one or more arms (542) may be different from other arms (542). For instance, in some versions, one or more arms (542) may have a longer or shorter length relative to another arm (542). In addition, or in the alternative, in some versions, one or more arms (542) may define a different shape relative to another arm (542). Still other variations of the configuration of each arm (542) relative to other arms (542) will be apparent to those of ordinary skill in the art in view of the teachings herein.

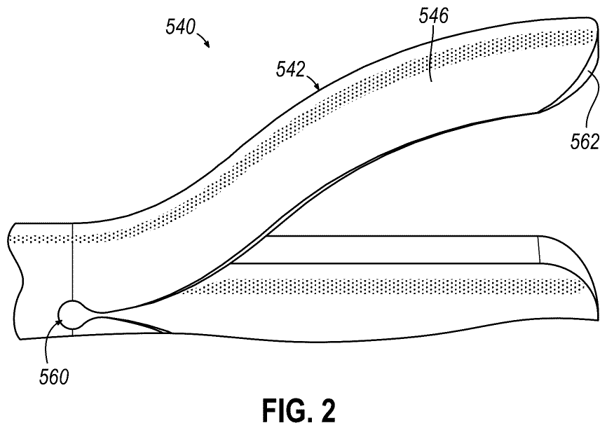

As noted above, arms (542) may move between a pre-deployment configuration and a post-deployment configuration. FIGS. 1 and 2 show arms (542) in the post-deployment state. In this state, each arm (542) curves outwardly from a longitudinal axis defined by marker element (512). In other words, arms (542) may be splayed outwardly relative to each other. In some versions, this outward extension, curvature, and/or splaying of arms (542) may double the diameter of marker (500) relative to the diameter when in the pre-deployment configuration. In other versions, this outward extension, curvature, and/or splaying of arms (542) may triple the diameter of marker (500) relative to the diameter when in the pre-deployment configuration. As best seen in FIG. 2, the particular outward curvature of each arm (542) may be non-linear with the curvature having a relatively low slope initially, followed by a relatively high slope, and then a tapering slope towards alignment with the longitudinal axis defined by marker element (512). In other word, the outward curvature of each arm (542) may initially define a relatively concave (relative to the exterior of a given arm (542)) section proximate base portion (520) and then transition to a relatively convex section proximate distal tip (562).

As described above, arms (542) of the present version are integral with base portion (520). Marker element (512) is thus a single monolithic, homogenous continuum of material. The particular material used in the present version is a shape-memory alloy. As will be described in greater detail below, the shape-memory characteristic of the material used in marker element (512) may be used to permit transitioning of arms (542) from the pre-deployment configuration to the post-deployment configuration using patient heat from tissue surrounding the biopsy site to initiate the transition.

It should be understood that a variety of suitable shape-memory alloys may be used for marker element (512). In addition, the constituents of any one shape-memory alloy may be varied to produce desired properties (e.g., transformation temperature). One suitable shape-memory alloy may include Nitinol (nickel-titanium). Any suitable grade of Nitinol may be used. By way of example only, one suitable grade of Nitinol may include Nitinol S (ASTM 2063). Alternatively, other shape-memory alloys may include copper-based alloys, gold-cadmium, silver-cadmium, and/or nickel-aluminum.

Returning to FIG. 1, the entire outer surface of marker element (512) may include a surface treatment (ST). To the extent surface treatment (ST) has the appearance in FIG. 1 as covering a portion of marker element (512), it should be understood that such appearance is for illustration purposes only and in some versions, surface treatment (ST) covers the entire surface of marker element (512). Surface treatment (ST) is generally configured to enhance the echogenicity of marker element (512). In the present version, surface treatment (ST) includes a roughened surface texture caused by sandblasting. However, it should be understood that in other versions, various alternative surface treatments (ST) may be used. For instance, in some versions, surface treatment (ST) may include an echogenic coating having a plurality of microspheres and a polymer binder. Such a coating may be in addition to, or in lieu of, sandblasting. In addition, or in the alternative, other versions may include a roughened surface by various processing mechanisms such as peening, griding, knurling, and/or etc. Of course, various alternative configurations of surface treatment (ST) may be used as will be apparent to those of ordinary skill in the art in view of the teachings herein.

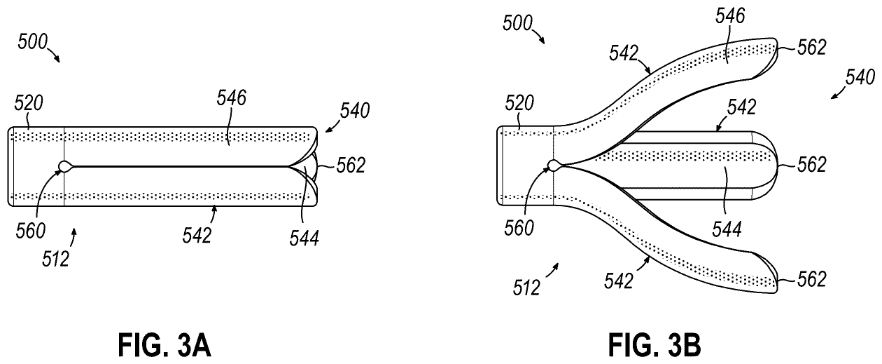

FIGS. 3A through 4 show aspects of a use of marker element (512) for marking tissue. As can be seen in FIG. 3A, marker element (512) may initially begin with arms (542) in the pre-deployment configuration. In the pre-deployment configuration, arms (542) may be compressed inwardly towards the longitudinal axis defined by marker element (512) so that each arm (542) abuts each adjacent arm (542). When compressed as shown, arms (542) form a generally hollow cylindrical shape corresponding to the shape of base portion (520). Thus, marker element (512) may define a generally cylindrical shape when arms (542) are in the pre-deployment configuration.

In some versions, the post-deployment configuration described above may correspond to a “natural,” “original,” “shape set,” or “undeformed” shape. In other words, the post-deployment configuration may be the natural or as-manufactured shape of arms (542) imparted into the shape-memory alloy. Arms (542) may then be compressed into the pre-deployment shape shown in FIG. 3A subsequently during manufacturing to prepare marker element (512) for use in deployment at a biopsy site.

Regardless of how the transformation of arms (542) to the pre-deployment configuration is performed, marker element (512) may be introduced into a patient at a biopsy site while in the pre-deployment configuration. The pre-deployment configuration may be desirable for introduction at the biopsy site because the compact and uniform shape may promote use with instruments similar to marker delivery device (150) described in greater detail below. The compact and uniform shape may also promote movement of marker element (512) through tissue for placement at the biopsy site.

Once marker element (512) is positioned as desired, marker element (512) may absorb heat from the tissue adjacent to the biopsy site and increase in temperature. The temperature of marker element (512) may increase until reaching a transformation temperature of the shape-memory alloy of marker element (512). Once the transformation temperature is reached, the alloy may return to its natural, original, or undeformed shape. In the present version, this shape corresponds to the post-deployment configuration of arms (542). Thus, heat from the patient may initiate a transition of arms (542) from the pre-deployment configuration to the post-deployment configuration.

As best seen in FIGS. 3B and 4, the transition to the post-deployment configuration includes arms (542) moving away from the longitudinal axis defined by marker element (512). In other words, arms (542) may spread or splay outwardly relative to each other. During this transition, arms (542) may engage the tissue surrounding the biopsy site to anchor marker element (512) in the tissue, thereby holding marker element (512) in position at the biopsy site.

### II. EXEMPLARY MARKER

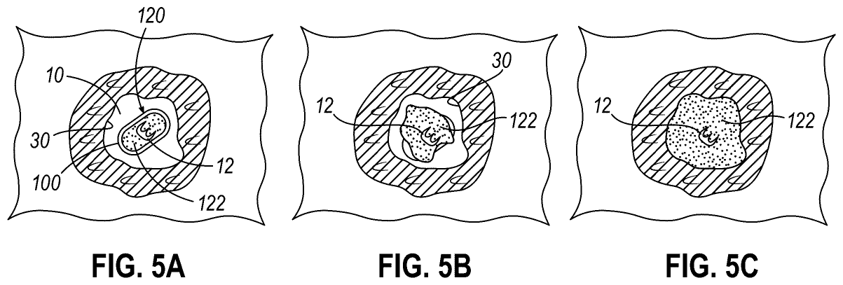

Aspects presented herein relate to devices and procedures for manufacturing a marker for percutaneously marking a biopsy cavity (10) having surrounding tissue (30), as shown in FIGS. 5A-5C. For instance, as seen in FIG. 5A, a marker (100) (or alternatively marker (500)) may be initially placed in the biopsy cavity (10) to facilitate relocation of the biopsy site. Although marker (100) is described separately herein, it should be understood that marker (100) may incorporate various features of marker (500) described above such as anchor portion (540), arms (542), base portion (520), and/or etc. Marker (100) may comprise a carrier (120) and a marker element (12). As noted above, carrier (120) may be readily incorporated into marker (500) or marker (100). Carrier (120) generally includes a bioabsorbable marker material (122). Thus, carrier (120) is generally configured for absorption into a patient after placement of marker (100) within the biopsy cavity (10). In some versions, carrier (120) can include a plurality of microbubbles to enhance visualization of carrier (120) under ultrasound. As will be described in greater detail below, marker material (122) is generally bioabsorbable such that marker material (122) may be generally absorbed into the patient's tissue over time. In the present version, marker material (122) comprises a hydrogel that is initially in a dehydrated state. Although a hydrogel is used in the present version, it should be understood that in other versions marker material (122) may comprise other known bioabsorbable materials.

In the present version, marker (100) further includes a marker element (12) that is generally not bioabsorbable (e.g., permanent or semi-permanent). Marker element (12) may comprise a radiopaque or echogenic marker embedded within the bioabsorbable marker material (122) of carrier (120). For instance, marker element (12) may comprise metal, hard plastic, or other radiopaque or hyperechoic materials known to those of ordinary skill in the art in view of the teachings herein. In other versions, marker (100) may be formed without a marker element (12). In still other versions, marker (100) may be formed with only marker element (12) such that carrier (120) is omitted and marker element (12) is in a “bare” form. In other words, in some versions marker (100) is formed of only marker element (12) as a bare clip or other structure and carrier (120) may be omitted entirely. Additionally, as noted above, in some versions, marker element (12) may include features of marker element (512) such as base portion (520), anchor portion (540), arms (542), and/or etc.

Marker material (122) is generally expandable once disposed within a patient at a biopsy site. As shown in FIGS. 5B and 5C, the initially dehydrated marker material (122) may absorb fluid from the surrounding tissue (30) into which it is inserted. In response to this absorption of fluid, maker material (122) may swell, thereby permitting carrier (120) to fill a cavity formed at a biopsy site by removal of tissue samples during a biopsy procedure. Biodegradable materials may be particularly suitable in applications where it is desired that natural tissue growth be permitted to completely or partially replace the implanted material over time. Accordingly, biocompatibility is ensured and the natural mechanical parameters of the tissue are substantially restored to those of the original condition.

Marker (100) (or alternatively marker (500)) may be inserted into the body either surgically via an opening in the body cavity (30), or through a minimally invasive procedure using such devices as a catheter, introducer or similar type insertion device. Such insertion may be immediately after removal of the tissue specimen using the same device used to remove the tissue specimen itself. Follow-up noninvasive detection techniques, such as x-ray mammography or ultrasound may then be used by the physician to identify, locate, and monitor the biopsy cavity site over a period of time via marker (100) (or alternatively marker (500)).

Marker (100) of the present version is large enough to be readily visible to a clinician under x-ray or ultrasonic viewing, for example; yet small enough to be able to be percutaneously deployed into the biopsy cavity and to not cause any difficulties with the patient. Although various aspects of marker (100) are described in connection with treatment and diagnosis of breast tissue, aspects presented herein may be used for markers in any internal, tissue, e.g., in breast tissue, lung tissue, prostate tissue, lymph gland tissue, etc.

The hydration of the marker material (122) of carrier (120) by the natural moisture of the tissue surrounding it causes expansion of the polymer and thus minimizes the risk of migration. The growing hydrogel-based marker material (122) centers marker (100) in the biopsy cavity as it grows. As the hydrogel expands, naturally present moisture from the surrounding tissue, the hydration enables increasing sound through transmission, appears more and more hypoechoic and is easy to visualize on follow up ultrasound studies.

The hydrated hydrogel marker material (122) of carrier (120) may also be used to frame permanent marker (12). The hypoechoic nature of the hydrated marker material (122) enables ultrasound visibility of the permanent marker (12) within the hydrogel hydrated marker material (122) because the permanent marker (12) is outlined as a specular reflector within a hypoechoic hydrated marker having a water-like nonreflective substrate.

### III. EXEMPLARY MARKER DELIVERY DEVICE

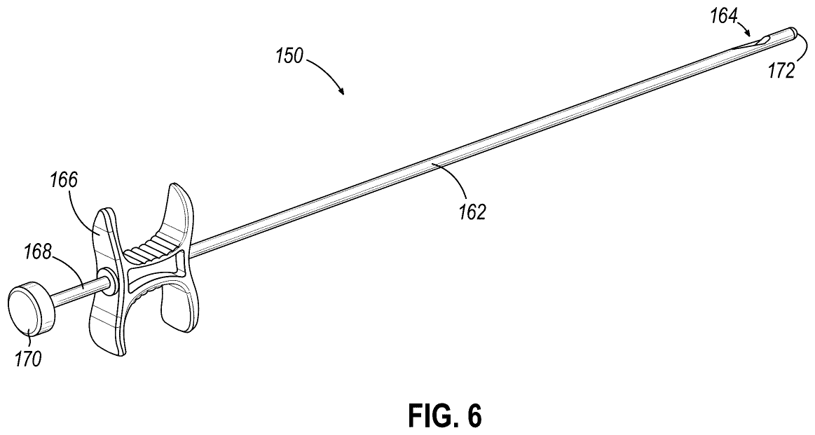

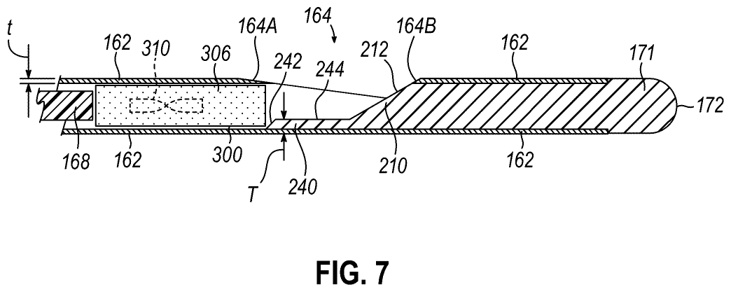

In some versions, it may be desirable to deploy any one or more of markers (100, 500) described above within the body cavity (30) using certain marker delivery devices. For instance, FIGS. 6 and 7 show an exemplary marker delivery device (150) which includes an elongate outer cannula (162) having a marker exit, such as side opening (164) formed adjacent to, but spaced proximally from, the distal end of the cannula (162).

A grip (166) can be provided at the proximal end of cannula (162). A push rod (168) can be provided, with push rod (168) extending coaxially in cannula (162) such that push rod (168) is configured to translate within cannula (162) to displace one or more markers through side opening (164) (see FIG. 7). Rod (168) may have sufficient rigidity in compression to push a marker from an internal lumen (165) of cannula (162) out through opening (164), yet be relatively flexible in bending. A plunger (170) is coupled at the proximal end of rod (168) for forcing rod (168) distally in cannula (162) to deploy a marker out of cannula (162).

A user may grasp grip (166) with two fingers, and may push on plunger (170) using the thumb on the same hand, so that marker delivery device (150) is operated by a user's single hand. A spring (not shown) or other feature may be provided about rod (168) to bias rod (168) proximally relative to grip (166) and cannula (162).

FIG. 7 shows a cross-sectional view of a distal portion of the marker delivery device (150). As can be seen, a biopsy marker (300) similar to markers (100, 500) described above is disposed within internal lumen (165) of cannula (162). Although marker (300) is shown in the present version, depiction of marker (300) is for illustration purposes only and in some versions markers (100, 500) may be used in addition to, or in lieu of, marker (300). In the present version, marker (300) may include a biodegradable or otherwise resorbable marker material (306), such as a generally cylindrically shaped body of collagen, hydrogel, or etc., and a metallic, generally radiopaque permanent marker or marker element (310) (shown in phantom) disposed within or otherwise carried by marker material (306).

Cannula (162) may be formed of any suitable metallic or non-metallic material. In some versions, cannula (162) is formed of a thin-walled hollow tube formed of a suitable medical grade plastic or polymer. One suitable material is a thermoplastic elastomer, such as Polyether block amide (PEBA), such as is known under the tradename PEBAX. Cannula (162) may be formed of PEBAX, and may be substantially transparent to visible light and X-ray.

Side opening (164) may be formed by cutting away a portion of the wall of cannula (162). Side opening (164) communicates with an internal lumen (165) of cannula (162). Side opening (164) may extend axially (in a direction parallel to the axis of lumen (165)) from a proximal opening end (164A) to a distal opening end (164B), as illustrated in FIG. 7.

In the present version, distal tip (172) extends from the distal end of cannula (162) and is rounded as shown in FIG. 7. Referring to FIG. 7, the distal end of cannula (162) is closed by a unitary endpiece (171), with a portion of endpiece (171) extending into internal lumen (165) of cannula (162). Endpiece (171) may be a molded or cast component. Endpiece (171) comprises a tip (172), a ramp (210) having a ramp surface (212), and a marker engaging element (240). Ramp surface (212) aids in directing marker (300) from internal lumen (165) through side opening (164). Marker engaging element (240) helps to retain marker (300) in internal lumen (165) until the user intends to deploy marker (300).

Marker engaging element (240) is disposed within internal lumen (165), and at least a portion of marker engaging element (240) is disposed distally of proximal end (164A) of side opening (164). Marker engaging element (240) extends along a portion of the floor of cannula (162) under opening (164) such that marker engaging element (240) is positioned to reinforce the portion of cannula (162) in which opening (164) is formed. For instance, by positioning marker engaging element (240) underneath opening (164), as shown in FIG. 7, element (240) helps to stiffen cannula (162) in the region where wall of cannula (162) is cut to form opening (164). As shown in FIG. 7, marker engaging element (240) extends from the proximal most portion of ramp surface (212), and does not extend proximally of side opening (164), though in other embodiments, a portion of element (240) may extend proximally of opening (164).

As shown in FIG. 7, marker engaging element (240) is in the form of a step having a generally uniform thickness (T) along element's (240) axial length, except that element (240) has a tapered proximal end (242). Tapered proximal end (242) forms an included angle with the longitudinal axis of lumen (165) (included angle with a horizontal line in FIG. 7) of about 45 degrees, while ramp surface (212) forms an included angle with the longitudinal axis of about 30 degrees. Of course, any number of other suitable angles may be used.

As shown in FIG. 7, an upwardly facing surface (244) (surface facing opening (164)) of marker engaging element (240) extends distally to contact ramp surface (212), so that there is not a space or gap between surface (244) and ramp surface (212). Such an arrangement is advantageous to reduce the possibility that marker (300), upon moving past marker engaging element (240), may become lodged between marker engagement element (240) and ramp (212). In some versions, marker engaging element (240), ramp (210), and/or tip (172) are formed of, or include, a material that is relatively more radiopaque than the wall of cannula (162). For instance, where element (240), ramp (210), and tip (172) are formed as an integral endpiece (171), endpiece (171) may include a radiopaque additive, such as barium sulfate. For instance, endpiece (171) may be a component molded of PEBAX, with about 20 percent by weight barium sulfate added to the molten PEBAX mold composition. The relatively more radiopaque marker engaging element (240), ramp (210), and tip (22) may be useful in distinguishing the position of those components using radiographic imaging. Also, where ramp (210) and/or step of engaging element (240) are positioned in association with opening (164), the addition of a radiopaque material can help identify the position of opening (164), and the position of marker (300) relative to opening (164) before, during, or after deployment of marker (300).

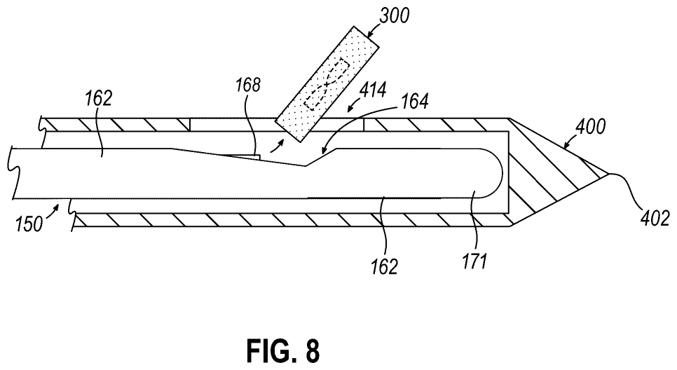

Referring to FIG. 8, marker delivery device (150) is used to deploy a marker (300) (or alternatively any one or more of markers (100, 500)) to mark a biopsy location within a patient. In FIG. 8, a cannular biopsy needle (400) is shown having a closed distal end with piercing tip (402) and a lateral tissue receiving aperture (414). Marker delivery device (150) is introduced to a biopsy site through biopsy needle (400), which may be the same needle (400) used to collect a tissue sample from the biopsy site. Biopsy needle (400) may be of the type used with single insertion, multiple sample vacuum assisted biopsy devices. Several such biopsy devices are disclosed in the various patents and patent applications that have been referred to and incorporated by reference herein, though other biopsy devices may be used.

FIG. 8 shows the distal end of marker delivery device (150) disposed within needle (400). Needle (400) may be positioned in tissue, and a biopsy sample may be obtained through lateral aperture (414), thereby providing a biopsy cavity adjacent lateral aperture (414). Then, after the tissue sample has been obtained and transferred proximally through needle (400), and without removing needle (400) from the patient's tissue, marker delivery device (150) is inserted into a proximal opening in needle (400). In FIG. 8, needle (400) and marker delivery device (150) are positioned such that opening (164) of cannula (162) and lateral aperture (414) of needle (400) are substantially aligned axially and circumferentially. Then, with marker delivery device (150) and needle (400) so positioned at the biopsy site, push rod (168) is advanced to deploy marker (300) (or alternatively any one or more of markers (100, 500)) up ramp surface (212), through opening (164), and then through lateral aperture (414), into the biopsy cavity.

### IV. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy site marker, comprising: a marker element, the marker element including a base portion and an anchor portion, the anchor portion extending distally from the base portion, the anchor portion including a plurality of arms, each arm of the plurality of arms being configured to be responsive to heat to transition from a pre-deployment configuration to a post-deployment configuration, each arm of the plurality of arms extending outwardly from a longitudinal axis defined by the marker element when the plurality of arms are in the post-deployment configuration.

### Example 2

The marker of Example 1, each arm of the plurality of arms being configured to abut one or more adjacent arms when in the pre-deployment configuration.

### Example 3

The marker of Examples 1 or 2, each arm of the plurality of arms being arranged in a circular pattern relative to the base portion.

### Example 4

The marker of any one or more of Examples 1 through 3, each arm having a curved inner surface and a curved outer surface.

### Example 5

The marker of Example 4, the curved inner surface and the curved outer surface of each arm of the plurality of arms being configured to form a hollow cylindrical shape when the plurality of arms are in the pre-deployment configuration.

### Example 6

The marker of any one or more of Examples 1 through 5, each arm of the plurality of arms defining a curved profile when in the post-deployment configuration, the curved profile having an initially increasing slope followed by a decreasing slope as each arm extends distally from the base portion.

### Example 7

The marker of any one or more of Examples 1 through 6, the marker element being configured to slidably engage a cannula when each arm of the plurality of arms are in the pre-deployment configuration.

The marker of any one or more of Examples 1 through 7, the plurality of arms including three arms.

### Example 9

The marker of any one or more of Examples 1 through 8, the marker element defining a plurality of relief openings, each relief opening being disposed between an arm of the plurality of arms, each relief opening being configured to permit movement of each arm relative to the base portion.

### Example 10

The marker of Example 9, each relief opening defining a tear-drop shape.

### Example 11

The marker of any one or more of Examples 1 through 10, each arm of the plurality of arms being integral with the base portion.

### Example 12

The marker of any one or more of Examples 1 through 11, the base portion having a hollow interior and an open proximal end, the open proximal end of the base portion being configured to promote tissue in-growth.

### Example 13

The marker of any one or more of Examples 1 through 12, the marker element including a surface treatment to one or more exterior surfaces of the marker element, the surface treatment being configured to increase echogenicity of the marker element.

### Example 14

The marker of Example 13, the surface treatment including a sand blasted surface.

### Example 15

The marker of any one or more of Examples 1 through 14, the marker element being configured as a bare marker element.

### Example 16

A biopsy site marker, the biopsy site marker comprising: a base portion; and an anchor portion extending from the base portion, the anchor portion including a plurality of outriggers, each outrigger having a shape set position and a compressed position, each outrigger being splayed from each other outrigger when in the shape set position, each outrigger being configured to return to the shape set position from the compressed position when the anchor portion is exposed to a predetermined temperature.

### Example 17

The biopsy site marker of Example 16, the marker defining a bell-shape when each outrigger is in the shape set position.

### Example 18

The biopsy site marker of Examples 16 or 17, the anchor portion defining a first diameter when each outrigger is in the compressed position, the anchor portion defining a second diameter when each outrigger is in the shape set position, the second diameter being at least double the first diameter.

### Example 19

The biopsy site marker of Examples 16 or 17, the anchor portion defining a first diameter when each outrigger is in the compressed position, the anchor portion defining a second diameter when each outrigger is in the shape set position, the second diameter being at least triple the first diameter.

### Example 20

A biopsy site marker, comprising: a marker element, the marker element defining a hollow cylindrical shape extending from a proximal end to a distal end, the marker element further defining a plurality slots extending from the distal end to a base portion to define a plurality of arms, each arm of the plurality of arms being configured to move outwardly from a longitudinal axis defined by the marker element in response to heat at a biopsy site.

### Example 21

The biopsy site marker of Example 20, the proximal end and the distal end of the marker element having rounded edges.

### Example 22

The biopsy site marker of Examples 20 or 21, the plurality of slots terminating in a relief opening, the relief opening being sized to permit movement of the arms relative to a portion of the marker element.

### Example 23

The biopsy site marker of any one or more of Examples 20 through 22, the marker element including a single material, the single material being Nitinol.

### V. CONCLUSION

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy site marker, comprising: a marker element, the marker element including a base portion and an anchor portion,
the anchor portion extending distally from the base portion, the anchor portion including a plurality of arms,
each arm of the plurality of arms being configured to be responsive to heat to transition from a pre-deployment configuration to a post-deployment configuration,
each arm of the plurality of arms extending outwardly from a longitudinal axis defined by the marker element when the plurality of arms are in the post-deployment configuration.

2. The marker of claim 1, each arm of the plurality of arms being configured to abut one or more adjacent arms when in the pre-deployment configuration.

3. The marker of claim 1, each arm of the plurality of arms being arranged in a circular pattern relative to the base portion.

4. The marker of claim 1, each arm having a curved inner surface and a curved outer surface.

5. The marker of claim 4, the curved inner surface and the curved outer surface of each arm of the plurality of arms being configured to form a hollow cylindrical shape when the plurality of arms are in the pre-deployment configuration.

6. The marker of claim 1, each arm of the plurality of arms defining a curved profile when in the post-deployment configuration, the curved profile having an initially increasing slope followed by a decreasing slope as each arm extends distally from the base portion.

7. The marker of claim 1, the marker element being configured to slidably engage a cannula when each arm of the plurality of arms are in the pre-deployment configuration.

8. The marker of claim 1, the plurality of arms including three arms.

9. The marker of claim 1, the marker element defining a plurality of relief openings, each relief opening being disposed between an arm of the plurality of arms, each relief opening being configured to permit movement of each arm relative to the base portion.

10. The marker of claim 9, each relief opening defining a tear-drop shape.

11. The marker of claim 1, each arm of the plurality of arms being integral with the base portion.

12. The marker of claim 1, the base portion having a hollow interior and an open proximal end, the open proximal end of the base portion being configured to promote tissue in-growth.

13. The marker of claim 1, the marker element including a surface treatment to one or more exterior surfaces of the marker element, the surface treatment being configured to increase echogenicity of the marker element.

14. The marker of claim 13, the surface treatment including a sand blasted surface.

15. The marker of claim 1, the marker element being configured as a bare marker element.

16. A biopsy site marker, the biopsy site marker comprising:
(a) a base portion; and
(b) an anchor portion extending from the base portion, the anchor portion including a plurality of outriggers, each outrigger having a shape set position and a compressed position, each outrigger being splayed from each other outrigger when in the shape set position, each outrigger being configured to return to the shape set position from the compressed position when the anchor portion is exposed to a predetermined temperature.

17. The biopsy site marker of claim 16, the marker defining a bell-shape when each outrigger is in the shape set position.

18. The biopsy site marker of claim 16, the anchor portion defining a first diameter when each outrigger is in the compressed position, the anchor portion defining a second diameter when each outrigger is in the shape set position, the second diameter being at least double the first diameter.

19. The biopsy site marker of claim 16, the anchor portion defining a first diameter when each outrigger is in the compressed position, the anchor portion defining a second diameter when each outrigger is in the shape set position, the second diameter being at least triple the first diameter.

20. A biopsy site marker, comprising: a marker element, the marker element defining a hollow cylindrical shape extending from a proximal end to a distal end,
the marker element further defining a plurality slots extending from the distal end to a base portion to define a plurality of arms,
each arm of the plurality of arms being configured to move outwardly from a longitudinal axis defined by the marker element in response to heat at a biopsy site.

20.-23. (canceled)
